(19) United States
(12) Patent Application Publication
(10) Pub. No.: US 2025/0152762 A1
(43) Pub. Date: May 15, 2025

(54) INTERMITTENT CATHETER STORAGE/STERILIZATION/WARMING

(71) Applicant: ConvaTec Limited, Flintshire (GB)

(72) Inventor: Manjunath Penagondla, Warrington (GB)

(21) Appl. No.: 19/019,940

(22) Filed: Jan. 14, 2025

Related U.S. Application Data
(63) Continuation of application No. PCT/GB2023/051883, filed on Jul. 18, 2023.
(60) Provisional application No. 63/390,338, filed on Jul. 19, 2022.

(30) Foreign Application Priority Data
Aug. 10, 2022 (GB) GB2211713.9

(51) Int. Cl. A61L 2/18

(52) U.S. Cl. CPC A61L 2/18; A61L 2202/122; A61L 2202/24

(57) ABSTRACT

A catheter storage device that has a catheter-receiving chamber for receiving a catheter and a catheter warming means, the catheter warming means configured to warm the catheter.

### TECHNICAL FIELD OF THE INVENTION

The present invention relates to the storage, warming and/or sterilisation of intermittent catheters (e.g. intermittent urinary catheters).

### BACKGROUND TO THE INVENTION

A catheter is a medical device comprising a hollow catheter tube designed for insertion into canals, vessels, passageways or body cavities to permit injection, drainage or withdrawal of fluids or substances therefrom, or to ensure said canals, vessels, passageways etc. remain open. Urinary catheters are designed for use for insertion into a user's bladder via the urethra to drain the bladder.

Catheters, especially intermittent urinary catheters, are typically single-use and are intended to be discarded once used. This can be undesirable as it generates unwanted waste. It is therefore advantageous to provide a re-usable catheter. In order to provide a catheter that is reusable, issues surrounding the storage and sterilisation of the catheter must be addressed.

In an attempt to achieve a means of sterilisation, some prior art solutions provide an outer case into which the catheter can be introduced along with a sterilising solution. Examples of such catheter products are shown in PCT applications No. PCT/US2020/036969 and PCT/US2020/039190. In order to ensure that the entire catheter is sterilised either a sufficient amount of sterilisation solution must be provided to fully immerse the catheter or the case must be agitated (i.e. by the shaking the case). However, such arrangements are not without their drawbacks; where the catheter is fully immersed in sterilising solution a large quantity of sterilising fluid is required and requires the user to recharge it between every use and there is a risk of spillage. When the case is agitated the user may not know when the catheter has been completely treated with the sterilising solution.

Furthermore, to maximise comfort and minimise the risk of trauma and/or infection, an outer surface of the catheter tube is typically wetted using a wetting agent prior to insertion by the user. In further developments, the catheter tube itself comprises, is integrated with or is coated with a hydrophilic component (e.g. a hydrophilic polymer) which serves to reduce friction further upon application of a wetting agent (e.g. water).

Some catheters may be supplied pre-wetted in a packaging, for instance, where the catheter is at least partially submerged within wetting agent within the packaging. Whilst this may ensure the catheter tube is adequately wetted prior to use, such arrangements suffer in that components of the catheter other than the catheter tube such as a gripper element or funnel can also become wetted. This has a detrimental effect of the experience of the user where it may become difficult to hold and direct the catheter tube as required. This is particularly problematic where the user is performing self-catheterisation. Further, having the catheter submerged may effectively reduce the shelf-life of the catheter due to long-term exposure of components of the catheter to moisture.

It is therefore seen advantageous to provide a catheter which may be wetted and/or sterilised at or immediately prior to the point of use.

It is an aim of an embodiment or embodiments of the invention to overcome or at least partially mitigate one or more problems with the prior art and/or to provide an improved intermittent catheter.

### SUMMARY OF THE INVENTION

The present invention provides a catheter assembly according to the appended claims.

A broad aspect of the present disclosure provides a catheter storage and warming device comprising: a catheter-receiving chamber and a warming means.

The catheter-receiving chamber may be for receiving a catheter. The catheter warming means may be configured to warm the catheter.

According to a first aspect of the invention there is provided a catheter storage device comprising a catheter-receiving chamber for receiving a catheter and a catheter warming means, the catheter warming means configured to warm the catheter.

Advantageously, by providing a means of warming the catheter prior to use the user experience is improved as a source of discomfort is reduced.

The catheter storage device may also comprise a sterilisation device. The sterilisation device may be an atomiser. The sterilisation device may be a vaporiser. The device may further comprise a liquid storage means. The liquid storage means may contain liquid. The atomiser may be configured to atomise the liquid to form an atomised fluid. The vaporiser may be configured to vaporise the liquid to form a vapourised fluid. The atomised fluid may be supplied to the catheter-receiving chamber. The vapourised fluid may be supplied to the catheter-receiving chamber. The liquid may be a sterilising fluid.

The atomiser may be an ultrasonic piezoelectric atomiser. The piezoelectric atomiser may operate at a frequency of between 100 and 200 kHz, preferably, between 140 and 160 kHz, more preferably 142 kHz. The ultrasonic piezoelectric atomiser may be a piezoelectric disc transducer.

Atomisation typically produces a suspension of liquid droplets, with a diameter in the order of 1 μm, in air, referred to as a “mist” or aerosol. The use of ultrasonic atomisation is advantageous as it requires less energy than alternatives such as thermal atomisation and can be electronically controlled. Furthermore, ultrasonic atomisation also atomises any active ingredients held in solution in the sterilisation fluid, allowing a broader range of antimicrobial compounds to be used. It also reduces the amount of sterilisation fluid or liquid required to sterilise the catheter.

The vaporiser may be a thermal vaporiser. The thermal vaporiser may comprise a resistive wire. The resistive wire may be configured to heat the sterilisation to form an aerosol of sterilisation fluid.

Vaporisation provides an alternative means of generating an aerosol of liquid droplets suspended in air.

The sterilisation fluid storage means may comprise a sterilisation fluid storage chamber. The sterilisation fluid storage chamber may be fluidly connected to the catheter-receiving chamber. The sterilisation fluid storage chamber may be transparent or translucent. The sterilisation fluid storage chamber may be subdivided by one or more baffles. The one or more baffles may have a hole therein. The holes may be aligned. Each subdivision may contain sufficient sterilisation fluid for a single sterilisation cycle.

The sterilisation fluid storage chamber may comprise an aperture. The aperture may provide fluid communication between the interior and exterior of the fluid storage chamber. The aperture may be in fluid communication with, optionally aligned with, the holes in the one or more baffles. The aperture may be sealed with a cap prior to use. The cap may be removable by the user prior to use. The aperture may be sealed with foil. The foil may be pierced by the catheter storage and sterilisation device during insertion.

The fluid storage chamber may comprise a fluid absorbing material. The fluid absorbing material may be arranged in each sub-division. The fluid absorbing material may extend through the holes in the baffles. The fluid absorbing material may extend to the aperture in the sterilisation fluid storage chamber. The fluid absorbing material may be a foam. The fluid absorbing material may be fibrous. The fluid absorbing material may provide a wicking action.

By providing a wicking material in the sterilisation fluid storage chamber, the risk of spillage of sterilisation fluid is reduced. Furthermore, the orientation of the operation of the device becomes less dependent upon the orientation of the device which is beneficial in a portable, handheld, device.

The sterilisation fluid storage means may be integrally formed in the device. The sterilisation fluid storage means may be removable from the device.

The sterilisation fluid storage means may further comprise a port to refill the sterilisation liquid. The port may comprise a one-way valve. The sterilisation fluid storage means may be refilled via a syringe.

The sterilising solution may be hypochlorous acid solution. The sterilisation fluid may be selected from one of the following: chlorine dioxide, sodium hypochlorite, sodium hydroxide, sodium chloride, chlorine, hydrogen peroxide, photosensitisers, chloroazodin, dichlordimethylhydantoin, permanganate, alcohols (e.g. ethanol and/or isopropanol), phenols (e.g. phenol, thymol and/or chloroxyphenol), aldehydes (e.g. glutaraldehyde and/or noxythiolin) and/or acids (e.g. acetic acid, citric acid, peracetic acid and/or diperoxy dodecanoic acid) or combinations thereof. For example, the sterilisation solution may comprise sodium hypochlorite with sodium chloride; or the sterilisation solution may comprise sodium hypochlorite with sodium hydroxide; or the sterilisation solution may comprise sodium hypochlorite with hypochlorous acid; or the sterilisation solution may comprise chlorine with hypochlorous acid.

The catheter-receiving chamber may be elongate. The catheter receiving chamber may be tubular. The catheter-receiving chamber may be formed from a transparent material. The catheter-receiving chamber may have an interior wall. The catheter-receiving chamber may have an exterior wall. The exterior wall of the catheter-receiving chamber may form the exterior wall of the catheter storage and sterilisation device. The exterior wall of the catheter-receiving chamber may comprise an exterior wall of an external housing. The catheter-receiving chamber may be formed from a translucent material. The catheter-receiving chamber may be formed of plastic. A first end of the catheter-receiving chamber may comprise an opening to allow insertion of the catheter. A second end of the catheter-receiving chamber may be in fluid communication with the sterilisation fluid storage means. The fluid communication between the catheter-receiving chamber and the sterilisation fluid storage means may be via the atomiser.

The catheter storage and sterilisation device may further comprise a catheter. The catheter may be a urinary catheter. The catheter may be an intermittent urinary catheter. The sterilisation (preferably the atomiser or vaporiser) device may be arranged proximate to an insertion end of the catheter.

The catheter-receiving chamber may comprise a catheter holder. The catheter holder may not engage with the catheter tube. The catheter holder may comprise one or more ribs, projections, pips, pins fins or the like. The catheter holder may comprise one or more ribs. The catheter holder may engage with the catheter funnel. The catheter holder may engage with the catheter connector. The catheter holder may retain the catheter centrally within the catheter-receiving chamber. The ribs may project radially inwards from the inner wall of the catheter-receiving chamber. The ribs may be configured to engage with the catheter at a connector arranged thereon. The catheter may further comprise a funnel. The catheter may further comprise a catheter tube. The connected may be arranged between the funnel and the catheter tube. The funnel and connector may be integrally formed. The funnel may comprise a textured exterior surface. The exterior surface may provide a handling surface. Advantageously, where the catheter holder attaches to the catheter at a point other than the catheter tube this ensures that the entire surface of the catheter tube can be sterilised.

The catheter-receiving chamber may be integrally formed with a sterilisation unit. The catheter-receiving chamber may be provided with a cap. The catheter-receiving chamber and cap may be attached with a snap fit, for example an annular snap fit. The catheter-receiving chamber and cap may be attached with a fixed fit. The catheter-receiving chamber and cap may be attached with a snap fit. The catheter-receiving chamber and cap may be attached with a screw fit. A seal may be provided between the catheter-receiving chamber and cap. The seal may be an O-ring. The seal may be an X-ring. The seal may be a U-cup seal. The catheter-receiving chamber may be configured such that upon removal of the cap, a handling surface on the catheter is exposed.

The catheter-receiving chamber may be detachable from a sterilisation unit. The detachable catheter-receiving chamber may be a cartridge. The cartridge may be formed of translucent material. The cartridge may comprise a catheter-receiving chamber. A/the housing may comprise a sterilisation unit.

The cartridge and housing maybe attached with a fixed fit. The cartridge and housing may be attached with a snap fit, for example an annular snap fit. The cartridge and housing may be attached with a screw fit. A seal may be provided between the cartridge and housing. The seal may be an O-ring. The seal may be an X-ring. The seal may be a U-cup seal.

The catheter-receiving chamber may be attached to the sterilisation unit by means of a fixed fit. The catheter-receiving chamber may be attached to the sterilisation unit by means of a snap fit, for example an annular snap fit. The catheter-receiving chamber may be attached to the sterilisation unit by means of a screw fit. A seal may be provided between the catheter-receiving chamber and the sterilisation unit. The seal may be an O-ring, an X-ring or a U-cup seal. The catheter-receiving chamber may be configured such that upon being detached from the sterilisation unit, a handling surface on the catheter is exposed.

Advantageously, the provision of a seal improves the time that the catheter-receiving chamber remains sterile after sterilisation. This means that in some instances the catheter may be sterilised some time prior to use, for example, when initially inserted, allowing the catheter to be deployed quicker (i.e. without running a sterilisation cycle immediately before use). For example, immediately after using a catheter, it may be inserted into the device and sterilised; it may then remain sealed in the device for several hours, potentially even overnight, until the user next needs to drain their bladder.

The catheter-receiving chamber may further comprise a UV LED. The catheter-receiving chamber may comprise a plurality of UV LEDs. The one or more LEDs may have a wavelength of between 200 and 300 nm.

Advantageously, the provision of a UV LED provides a secondary means of sterilising the catheter.

The catheter warming means may be a heater. The catheter warming means may be a heating element. The heating element may be a resistive wire heater. The resistive wire may be provided on a wall of the catheter-receiving chamber, for example on the interior wall of the catheter-receiving chamber. The resistive wire may be arranged around the catheter-receiving chamber. The resistive wire may be coiled around the interior wall of the catheter-receiving chamber. The resistive wire may be wound helically around the interior wall. The resistive wire may be arranged radially outward of the catheter. The catheter warming means may be chemically activated. For example, the catheter warming means may comprise a chemical mixture, which is activatable to create an exothermic reaction. Various approaches are known for this, as are well known in the field of disposable hand warmers. For example, the chemical mixture may be air-activated (based on iron or a nanoparticle composite of zinc and carbon) or may use a supersaturated solution (such as sodium acetate solution).

By providing the resistive wire helically around the interior wall of the catheter-receiving chamber the entire surface of the catheter can be warmed. This is particularly useful as catheters a typically comprised of plastics which are poor thermal conductors, applying heat to the entire surface ensures the entire catheter is warmed.

Accordingly, in a preferred embodiment of the invention there is provided a catheter storage device comprising a catheter-receiving chamber for receiving a catheter and a catheter warming means, the catheter warming means configured to warm the catheter, wherein the warming means comprises a resistive wire heater wound helically around an interior wall of the catheter-receiving chamber.

The catheter storage device may be configured to sterilise the catheter prior to warming. The sterilisation and warming may be initiated simultaneously.

The catheter-receiving chamber may further comprise a sensor. The sensor may be configured to detect the presence of a catheter within the catheter-receiving chamber. The sensor may be a proximity sensor. The sensor may be an ultrasonic proximity detector. The sensor may be an optical sensor. The catheter storage and sterilisation device may be configured to be activated in response to a signal from the sensor. The atomiser or vaporiser may be configured to be activated when the catheter is inserted into the catheter-receiving chamber. The sensor may be configured to detect when the catheter is inserted into the catheter-receiving chamber. The sensor may be configured to activate the catheter warmer. The sensor may be configured to detect when the catheter is inserted into the catheter-receiving chamber and in response to activate the catheter warmer. The sensor may be configured to activate the atomiser or vaporiser. The sensor may be configured to detect when the catheter is inserted into the catheter-receiving chamber and in response to activate the atomiser or vaporiser.

The catheter-storage and warming device may further comprise a switch. The warming means may be configured to be activated by the switch. The atomiser or vaporiser may be configured to be activated by the switch. The switch may be configured to only be activatable a predetermined number of times. The switch may be a push button. The predetermined number of activations may be controlled by a PCB controller. The switch may comprise a mechanical latching wheel. The mechanical latching wheel may determine the predetermined number of activations. The switch may be resettable (i.e. the counter for the number of times the device has been activated may be reset to zero). The predetermined number of activations may be at least 2, 3, 4, 5, 6, 7, 8, 9 or 10. The predetermined number of activations may be no more than 20, 19, 18, 17, 16, 15, 14, 13, 12, 11, 10, 9, 8, 7, 6 or 5. For example, the predetermined number of activations may be between 5 and 10, for example 7. This would accord with approximately the number of times a catheter might need to be used (and hence sterilised) over the course of a single day.

Although the sterilisation allows the catheter to be used more than once, catheters may still have a finite lifespan. By including a means of monitoring this and/or limiting the number of activations, the case of use for the user is improved and chance of over-use is therefore reduced. By providing a means of resetting the counter, the catheter storage and sterilisation device can be used over the lifespan of multiple catheters, thereby reducing waste.

The catheter-storage and warming device may further comprise one or more indicators. The or an indicator, for example a first indicator may indicate that the device is operational, for example that the device is sterilising. The or an indicator, for example a second indicator may indicate that the device has completed the sterilisation. The indicators may be LEDs. The indicator may be an LCD display. Where the device has a predetermined number of activations, the LCD display may indicate the status thereof.

The catheter storage and warming device may further comprise a sterilising unit. The sterilising unit may comprise an electronics package. The electronics package may comprise a PCB controller. The PCB controller may comprise a timer. The PCB controller may comprise a counter. The PCB controller may be operably connected to the atomiser or vaporiser. The PCB controller may be operably connected to the UV LED. The PCB controller may be operably connected to the catheter warming means. The atomiser or vaporiser, and catheter warming means may be initiated by a single activation of the switch. The PCB controller may be operably connected to the sensor. The PCB controller may comprise a Bluetooth module. The timer may be configured to deactivate the atomiser or vaporiser after a predetermined time. The timer may be configured to deactivate the catheter warming means after a predetermined time.

The electronics package may comprise a battery. The battery may be rechargeable. The battery may be recharged via a USB connection.

By providing the device with a battery the portability of the device is improved. A user can warm the catheter at a convenient time and place.

The catheter storage and sterilisation device may further comprise a sterilisation unit. The sterilisation unit may comprise one or more of the following: the sterilisation fluid storage means, A sterilisation device (i.e. an atomiser, a vaporiser), the battery, the PCB controller, the or more indicators. An external wall of the sterilisation unit may comprise an external wall of an external housing of the device. The sterilisation unit may be elongate. The sterilisation unit may be tubular. The sterilisation unit may have the same cross-section as the catheter-receiving chamber.

The catheter storage and warming device may comprise a plurality of catheter-receiving chambers. Each catheter-receiving chamber may be in fluid communication with a separate sterilisation fluid storage chamber. Fluid communication with the separate sterilisation fluid storage chamber may be via separate atomisers or vaporisers. The plurality of catheter-receiving chambers may alternatively be in fluid communication with a single sterilisation fluid storage chamber. The plurality of catheter-receiving chambers may be in fluid communication with a single sterilisation fluid storage chamber via a single atomiser or vaporiser. Alternatively, each of the plurality of catheter-receiving chambers may be associated with a separate atomiser or vaporiser, each atomiser or vaporiser in fluid communication with a single sterilisation fluid storage chamber.

Each of the plurality of catheter-receiving chambers may be associated with a single warming means. Each catheter-receiving chamber be me associated with a separate warming means.

The catheter storage and warming device may comprise an external housing. The catheter may be a female intermittent catheter. The catheter may have a length of between 90 mm to 200 mm. The catheter may have a length of between 100 mm and 150 mm or for example between 130 mm and 155 mm, such as about 135 mm.

The external housing may have an elongate shape. The external housing may have a tubular shape. The external housing may have a length of between 150 mm to 300 mm. The catheter-receiving chamber may define a portion of the external housing. The sterilisation unit may define a portion of the external housing.

The external housing may be manufactured, imported and sold independently of the catheter. The catheter assembly may comprise a catheter.

The main body and/or cap may be rigid. The rigidity of the main body and cap may be configured to be resiliently deformable to aid a fixed fit of the cap on the main body.

The external housing may be comprised of plastic. The external housing may be comprised of thermoplastic. The cap and main body may be comprised of different materials. The external housing (optionally the cap or main body) may be comprised of polycarbonate; the external housing (optionally the cap or main body) may be comprised of polyethylene; the external housing (optionally the cap or main body) may be comprised of nylon.

The catheter comprises a catheter tube and an outlet body. The catheter may comprise an insertion end for inserting into the urethra and an outlet end from which fluid is drained during use. The outlet end may comprise one or more flow enhancing features such as a funnel which diverges along the flow direction. The outlet end may comprise an external handling surface. The external handling surface may be exposed for a user to handle when a cap is removed. The external handling surface may be exposer for a user to handle when the catheter-receiving chamber is removed. The external handling surface may comprise one or more surface features to enhance a user's grip. The one or more surface features may comprise one or more grooves. The catheter tube may comprise one or more inlets for receiving urine at an insertion end thereof.

The catheter tube may be functionalised. For example, it may comprise, be integrated with or be coated with a hydrophilic component (e.g. a hydrophilic polymer). The hydrophilic component serves to reduce friction further upon application of the wetting agent. At least an external surface of the catheter tube may be functionalised, e.g. the hydrophilic component may be provided on at least an external surface of the catheter tube (which is in contact with the urethra in use). The catheter may comprise a main flow path for the passage of urine. The main flow path may extend along and define a longitudinal axis of the catheter. The main flow path may be provided by a wall of catheter tube. The main flow path may have a proximal inlet at an insertion end of the catheter, and a distal outlet.

The catheter may comprise an outlet body. The outlet body may incorporate the terminal end of the catheter tube. The outlet body may comprise the external handling surface of the catheter. The outlet body may comprise one or more flow enhancing features for aiding the flow from catheter tube. The one or more flow enhancing features may comprise a funnel, for example.

The outlet body may comprise or be referred to as a connector which connects the outlet end, e.g. a funnel and/or the external handling features, and the catheter tube.

Optional features set out above may apply to any aspect of the invention described below. Thus, for example, the type of atomiser and sterilisation fluid are only described once above, but apply to all aspects and combinations of aspects and other optional features. Equally optional features set out below may apply to the above aspect of the invention or any further aspects disclosed below.

According to a second aspect of the invention there is provided a method of warming a catheter; the method comprising inserting a catheter into a catheter storage device, activating a catheter warming means, and warming the catheter.

The catheter storage and warming device may be the catheter storage and warming device according to the first aspect of the invention, which may optionally include any of the optional features set out above.

The method may further comprise activating an atomiser or a vaporiser, atomising or vaporising a sterilisation fluid, and directing the atomised or vaporised sterilisation fluid onto the catheter to sterilise the catheter.

The method may further comprise removing the catheter from the catheter-receiving chamber after sterilising the catheter. The catheter may be removed by the user handling a catheter handling means on the catheter.

The method may further comprise introducing the sterilised catheter into a urethra.

The catheter storage device may further comprise a sterilisation device. The method may further comprise sterilising the catheter. The sterilisation of the catheter may be performed prior to the step of warming the catheter.

The method may further comprise removing the catheter from the catheter-receiving chamber after warming the catheter. The catheter may be removed by the user handling a catheter handling means on the catheter.

The method may further comprise introducing the warmed catheter into a urethra.

According to a third aspect of the invention there is provided a catheter storage and sterilisation device; the device comprising a catheter-receiving chamber for receiving the catheter; a sterilisation fluid storage means for storing sterilisation fluid and an atomiser or vaporiser configured to atomise or vaporise the sterilisation fluid to form an atomised or vaporised sterilisation fluid and to supply the atomised or vapourised sterilisation fluid to the catheter-receiving chamber.

According to another broad aspect of the invention there is provided a method of wetting (e.g.) sterilising a catheter, the method comprising; inserting a catheter into a catheter storage, sterilisation or wetting device, activating an atomiser or a vaporiser, atomising or vaporising a fluid (for example a sterilisation fluid), and directing the atomised or vaporised fluid onto the catheter to wet the catheter. The fluid could be a lubrication fluid, such as water to activate a hydrophilic surface of a catheter.

According to a fourth aspect of the invention there is provided a method of sterilising a catheter, the method comprising; inserting a catheter into a catheter storage and sterilisation device, activating an atomiser or a vaporiser, atomising or vaporising a sterilisation fluid, and directing the atomised or vaporised sterilisation fluid onto the catheter to sterilise the catheter.

According to a fifth aspect of the invention there is provided a catheter storage and sterilisation device comprising a catheter-receiving chamber for receiving a catheter, a sterilisation mechanism and a sensor, wherein the sensor is configured to activate the sterilisation mechanism in response to detecting the introduction of a catheter into the catheter-receiving chamber.

According to a sixth aspect of the invention there is provided a method activating a sterilisation mechanism; the method comprising providing a catheter storage and sterilisation device comprising a sensor and sterilisation mechanism, and a catheter; inserting the into the catheter storage and sterilisation device, the sensor detecting the presence of the catheter, in response the sensor activating the sterilisation mechanism, and the catheter being sterilised.

The sterilisation means may be an atomiser. The sterilisation means may be a vaporiser.

The catheter storage and sterilisation device may be the catheter storage and sterilisation device according to the first aspect of the invention and the catheter storage and sterilisation device may optionally include any optional features outlined above.

The method may further comprise removing the catheter from the catheter-receiving chamber after sterilising the catheter. The catheter may be removed by the user handling a catheter handling means on the catheter.

The method may further comprise introducing the sterilised catheter into a urethra.

According to a seventh aspect of the invention there is provided a catheter storage and sterilisation device, the device comprising a housing and a removable cartridge; the cartridge including a catheter-receiving chamber for receiving a catheter; the cartridge being removably attachable to the housing and having an opening in fluid communication with the housing; the housing comprising a chamber for receiving a sterilisation fluid and a mechanism for supplying the sterilisation fluid to the cartridge via the opening.

According to an eighth aspect of the invention there is provided a method assembling a catheter storage and sterilisation device comprising a cartridge, a housing and a catheter, wherein the housing comprises a chamber for receiving a sterilisation fluid and a mechanism for supplying the sterilisation fluid to the cartridge; the method comprising; inserting the catheter into the cartridge and then attaching the cartridge to the housing.

The catheter storage and sterilisation device may be the catheter storage and sterilisation device according to the first aspect of the invention. The housing may be a sterilisation unit according to the first aspect of the invention. The mechanism may be the atomiser according to the first aspect of the invention. The mechanism may be the vaporiser according to the first aspect of the invention. Of course, the catheter storage and sterilisation device may optionally include any optional features outlined above.

The method may further comprise the step of activating the mechanism. The method may further comprise the step of detaching the cartridge from the housing. The method may further comprise the step of removing the sterilised catheter from the cartridge. The method may further comprise introducing the sterilised catheter into a urethra.

### DETAILED DESCRIPTION OF THE INVENTION

In order that the invention may be more clearly understood one or more embodiments thereof will now be described, by way of example only, with reference to the accompanying drawings, of which:

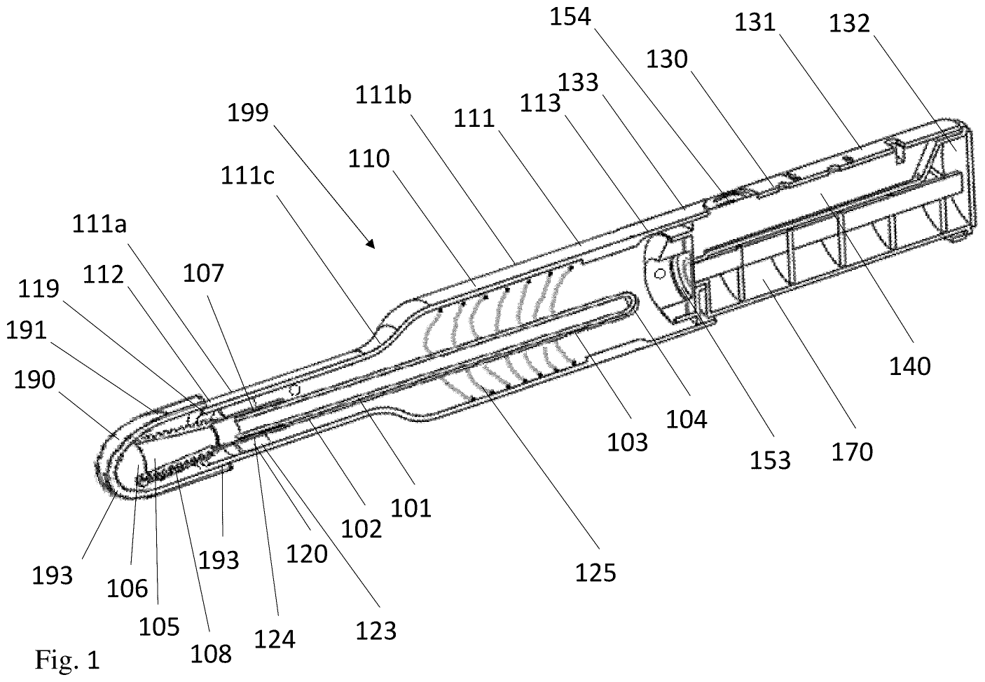

FIG. 1 shows a cross sectional schematic view of a catheter storage and warming device according to an embodiment of the disclosure;

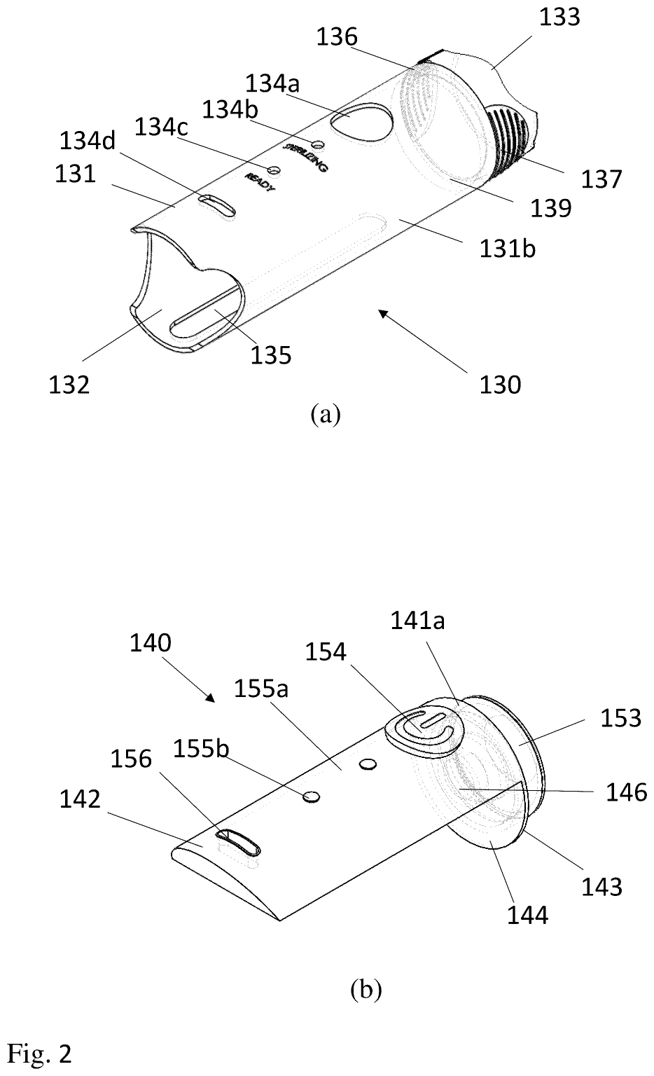

FIG. 2 shows an exploded perspective view of the sterilisation unit and electronics package of the catheter storage and sterilisation device of FIG. 1;

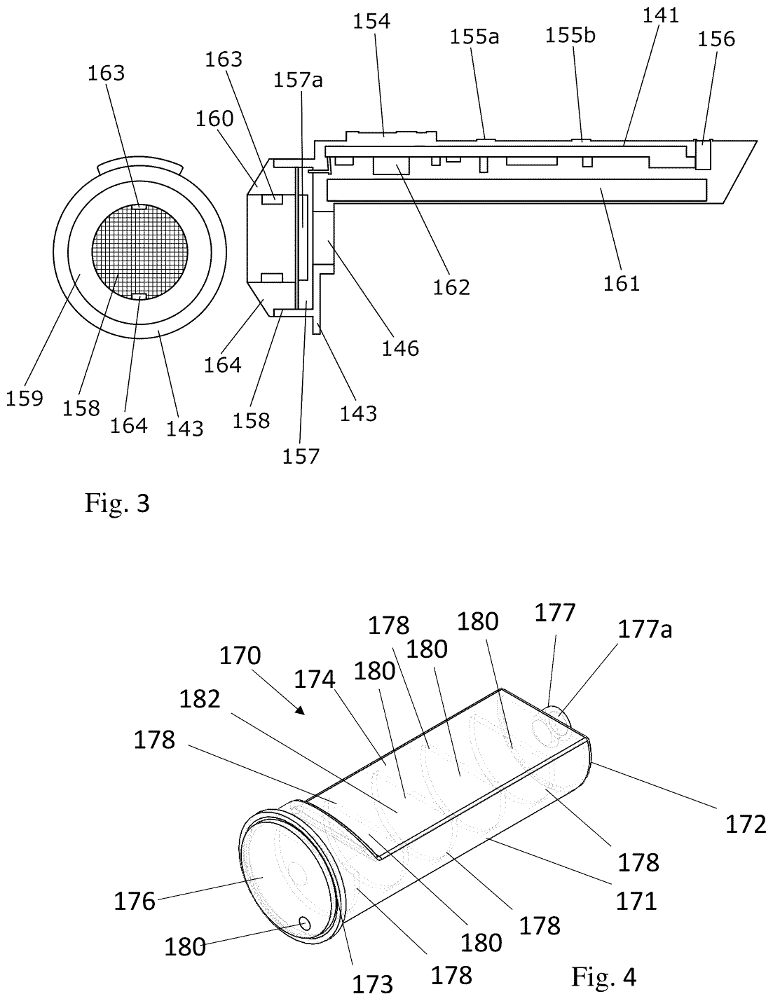

FIG. 3 shows a cross-sectional schematic view of the electronic components of the catheter storage and warming device of FIG. 1;

FIG. 4 shows a perspective schematic view of the sterilisation fluid storage chamber of the catheter storage and warming device of FIG. 1;

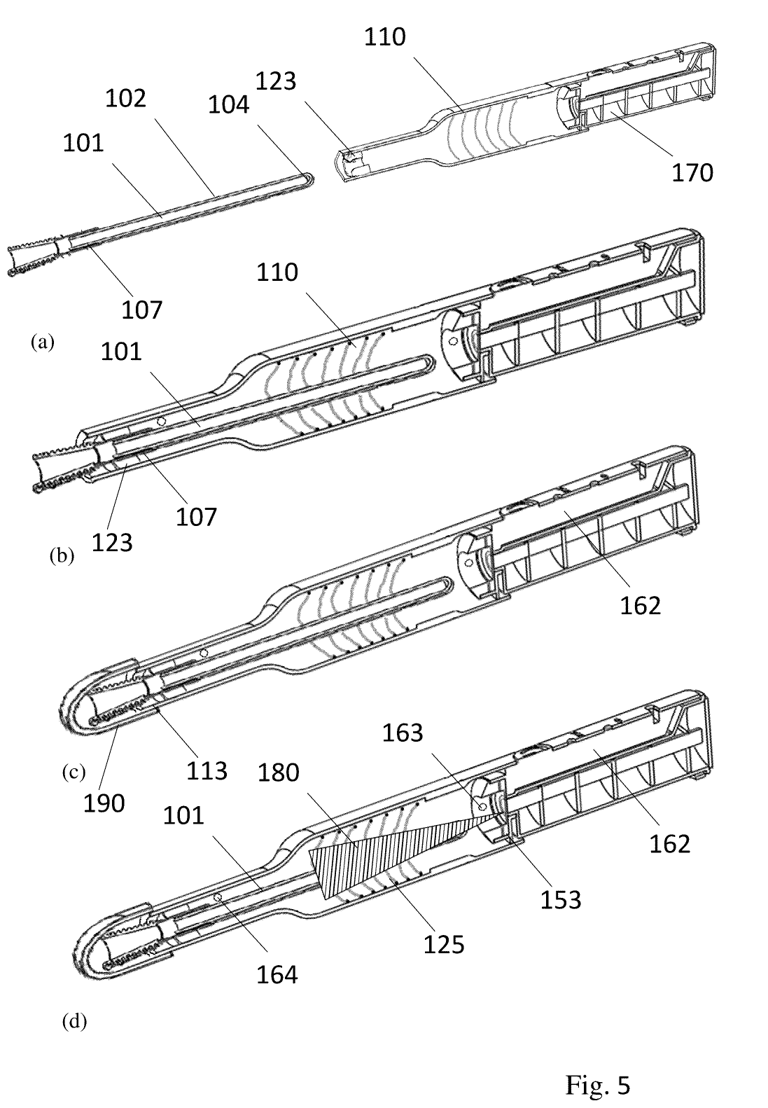

FIG. 5 shows a perspective view of the assembly and use of the device of FIG. 1;

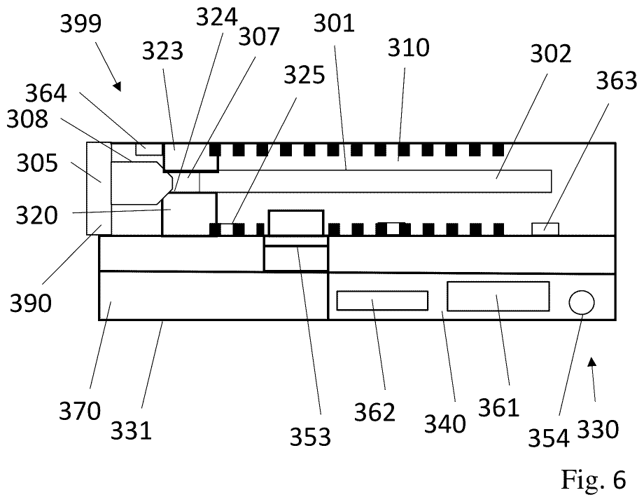

FIG. 6 shows a cross-sectional schematic view of a catheter storage and warming device according to a second embodiment of the disclosure;

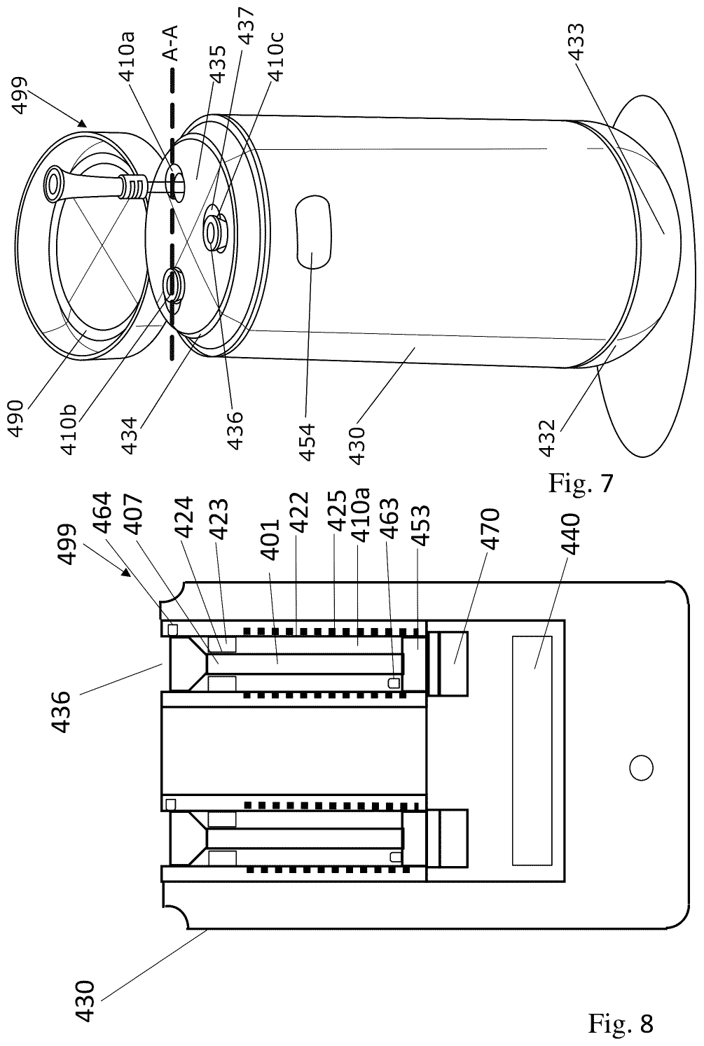

FIG. 7 shows a perspective view of a catheter storage and sterilisation device according to a fifth embodiment of the disclosure;

FIG. 8 shows a cross-sectional view of the catheter storage and sterilisation device of FIG. 8.

The one or more embodiments are described above by way of example only. Many variations are possible without departing from the scope of protection afforded by the appended claims.

FIG. 1 shows a cross sectional perspective view of an alternative catheter storage and warming device 199 according to a first embodiment of the present invention.

The catheter storage and sterilisation device 199 comprises a sterilisation unit 130 with an integrally formed catheter-receiving chamber 110.

The catheter-receiving chamber 110 is defined by the interior of an elongate plastic tube 111 with an open, distal, end 112 and a closed, proximal end 113. Arranged within the catheter-receiving chamber 110 is a catheter holder 120. Also shown is a catheter 101, the catheter 101 comprising a catheter tube 102 with a hole 103 arranged near the distal end 104 and a funnel 105 arranged at the proximal end 106. The funnel 105 is attached to the catheter tube 102 via a connector 107.

Arranged at the proximal end 113 of the catheter-receiving chamber 110, and forming the end of the enclosure, the sterilisation unit 130 comprises a tubular steriliser body 131 with an open, distal, end 132 and an opposing proximal end 133. Within the steriliser body 131 there is provided electronics package 140, sterilisation fluid storage chamber 170 and piezoelectric atomiser 153. Arranged on the outer surface of the steriliser body is a switch 154 operably connected to the electronics package 140 to control the sterilisation unit 130.

The tube 111 which forms the body of the catheter-receiving chamber 110 is, in this particular embodiment formed with two diameters, a first section 111a proximate to the open, distal, end 112 and a wider second section 111b proximate to the proximal end 113, the two sections 111a,b are of approximately equal length and joined by a tapered section 111c. The cartridge 110 is formed by moulding from a semi-translucent material.

At the open, distal end 112 of the catheter-receiving chamber 110 there is provided an engaging means 119 on the outer surface, to engage with a cap 190 as will be expanded upon below.

Within the narrow first section 111a of the catheter-receiving chamber there is provided a catheter holder 120 which comprises a short section of tubular plastic 21 with an outer diameter corresponding to the inner diameter of the second section 11b of the chamber 110 and radially nestled within. On the interior surface of the catheter holder 120 are provided three ribs 123 arranged equidistantly around the circumference, the ribs 123 extend parallel to the rotational axis of symmetry of the catheter holder and project radially inwards. The radially inward facing edges of the ribs 123 define engaging surfaces 124 which engage with the catheter 101, as such that a circle defined by the engaging surfaces is approximately equal to the outer diameter of the connector 107.

Within the wider second section 111b of the catheter-receiving chamber there is provided a resistive wire heater 125, the resistive wire heater is coiled helically around the interior surface of the chamber and is connected to the electronic package 140.

Also shown in FIG. 1 is the cap 190, the cap 190 has a tubular main body 191 with an internal diameter equal to the external diameter of the first section of the catheter-receiving chamber. The cap 190 has an open first end 192 and a closed second end 193, the closed end is closed by a hemispherical portion.

The catheter 101 is in this instance a female urinary catheter. The catheter comprises an elongate tubular portion 102, the catheter tube 102 is closed at the distal end 104 with a hemispherical cap. Proximate to the distal end there is provided a hole 103 which allows fluid communication between the outside of the catheter tube 102 and the hollow interior. The funnel 105 is provided at the proximal end 106 of the catheter tube, the funnel 105 is attached to the catheter tube 102 via a connector 107, the connector 107 is integrally formed with the funnel 105. The funnel and connector are hollow, and the interior surface of the connector connects to the exterior surface of the catheter tube, e.g. via a fixed fit. The funnel 105 has an open end in fluid communication with the opening 103 via the interior of the catheter tube to allow fluid to drain during use. The funnel 105 also has a textured outer surface 108 such that it can provide grip to a user when used as a handling means 108. The outer surface of the catheter tube 102 may be functionalised, for example it may be made from or coated in a water activated lubricant and/or antimicrobial material.

Whilst the embodiment is directed at a female intermittent urinary catheter, with an exemplary length of between 90 mm to 200 mm e.g. between 130 mm and 155 mm, such as about 135 mm, it is considered that teachings could be applied to male urinary intermittent catheters (which are typically longer) or even other types of catheter.

With reference to FIGS. 2 and 3 the sterilisation unit 130 is shown in greater detail. The steriliser body 131 is, in this embodiment, formed of a translucent plastic and is a continuation of the catheter-receiving chamber 110 (not shown). It is hollow with two open ends 132,133. The proximal end 133 continues into the second portion 111b of the catheter-receiving chamber 110. Provided on opposing sides of the step are two semi-circular gripping features 137.

The steriliser body 131 is provided with four holes 134 through the body 131 and arranged parallel to the longitudinal axis of the body 131, the four holes include a first hole 134a for the switch 154, a second hole 134b and a third hole 134c for indicia, and a fourth hole 134d for a USB charger, the details of which will be expanded upon below. Opposite the four holes 134 there is provided a slot 135 though the steriliser body 131 from the exterior to the interior, the slot 135 extends parallel to the longitudinal axis of the body 131.

On the interior surface of the steriliser body 31 radially inwards of the gripping features 137 is provided an annular projection 139 projecting radially inwards, the annular projection provides a stop 139 for the electronics package 140 and sterilisation fluid storage chamber 170 as will be explained in greater detail below.

With reference to FIGS. 2 and 3, the electronics package 140 is described in greater detail. The electronics body 141, in this exemplary embodiment has an overall prism shape with an arc cross section such that the curved surface 142 has a radius equal to the radius of the inner face of the steriliser body 131. One end 142 of the electronics body 141 is provided which a disk-shaped extension 143 arranged perpendicular to the long axis of the electronics body 141 such that the planar face 144 of the extension is normal to the electronics body 141, the disk 143 is sized to complete the partial circle of the arc of the electronics body 141. A circular aperture 146 is provided in the centre of the disk 143.

The piezo-electric atomiser 153 is provided on the face of the disk 143 facing away from the electronics body 141. The piezo-electric atomiser comprises an annular ceramic piezo-electric element 157, the aperture is covered by a stainless-steel mesh 158 attached to the face of the annular ceramic piezo-electric element. Arranged on the opposite side of the stainless-steel mesh 158 is a droplet guide flange 159, the droplet guide flange 159 is a tubular component, the longitudinal axis of which is normal to the plane of the stainless-steel mesh 158. The end of the droplet guide flange 159 distal from the mesh 158 is tapered on its radially outer edge, the tapered edge 160 aids with assembly as will be outlined below. The disk 143, disk aperture 146, piezo-electric atomiser aperture 157a, stainless-steel mesh 158 and droplet guide flange 159 are all co-axially arranged.

In some embodiments the piezo-electric atomiser may be replaced with a thermal vaporiser. The thermal vaporiser comprises a coil of thin wire (i.e. sufficiently thin that it heats up to the vaporisation temperature of the sterilisation fluid) arranged between the catheter-receiving chamber and the sterilisation fluid storage chamber.

On the inner wall of the droplet guide flange 159 is provided a UV LED 163 and a proximity sensor 164. The wavelength of the LED 163 is selected such that it has antimicrobial properties, for example it has a wavelength of 200 nm to 300 nm, and is arranged such that the emitted light is directed at the catheter tube.

The proximity sensor 164 is configured to detect the presence of the catheter 101 within the catheter-receiving chamber 110. The proximity sensor may be any suitable proximity sensor, for example an ultrasonic proximity sensor, optical or a capacitance proximity sensor. In other embodiments alternative types of sensors may be used to directly (i.e. through contact) or indirectly (i.e. remotely) determine the presence of the catheter 101, for example a micro switch or detector switch.

The electronics body 141 contains the electronic components of the device, in this particular embodiment, on the curved surface 142 is mounted the switch 154, two indicator lights 155a,b and a USB C charger port 156d. Internally the body comprises a battery 161 and a PCB controller 162. In some embodiments recharging the battery 161 may be done via wireless charging, in such embodiments the USB C charger port 156 may be removed.

The electronics package 140 is inserted into the steriliser body 131 via the opening at the distal end 132 of the steriliser body 131. The electronics package 40 is inserted with its droplet guide flange 159 first, with the tapered edge 160 helping to guide the electronics package 140 into the steriliser body 131. The electronics package 140 is inserted until the disk 143 abuts the stop 139 on the interior of the steriliser body 131, the electronics package 140 and steriliser body 131 are rotationally aligned such that the switch 154, indicator lights 155a,b and USB charger port 156 align with the corresponding holes 134a,b,c,d in the steriliser body 131.

With reference to FIG. 4, the sterilisation fluid storage chamber 170 of this particular embodiment is described in detail. The sterilisation fluid storage chamber 170 is defined by an enclosure 171 formed by a tubular body with a closed proximal end 172 and an open distal end 173 and is, in this embodiment formed of a semi-translucent plastic. An arc section conforming in size to the electronics body 141, extending from the proximal end is omitted thereby providing the enclosure with a flat face 174, the flat face 174 does not extend the entire length of the enclosure thus at the distal end 173 of the enclosure the enclosure has a circular cross-section.

A collar 175 is provided projecting radially outwards proximate to the distal end 1173 of the enclosure. The distal end 173 is closed by an end cap 176, provided as a separate component for ease of assembly and press fit into place. In some embodiments the centre of the distal end 173 there may be provided with a loading/reloading port 180 which extends into the interior of the enclosure and is sealed with a one-way valve.

The proximal end 172 of the enclosure of this embodiment is provided with a tip 177, the tip 177 is a tubular protrusion extending along the longitudinal axis of the enclosure away from the enclosure and arranged co-axially with the enclosure. The tip 177 comprises an aperture 177a providing fluid communication with the interior of the enclosure 171.

The interior of the sterilisation fluid storage chamber 170 is provided with baffles, in this case five baffles 178 arranged normal to the longitudinal axis of the chamber 170, the baffles 178 separating the chamber 170 into (six) separate sections 179. Fluid communication between the separate sections 179 is provided by a hole 181 in each baffle 178, each hole 80 is aligned with the aperture 177a in the tip 177. A cylindrical fibre reed 182 is inserted through these holes extending through along the longitudinal length of the chamber 170 and to the aperture 177a of the tip 177. In some embodiments the baffles may be omitted. In further embodiments the sterilisation fluid storage chamber 70 may be provided with an absorbent foam filling in place of the fibre reed and, optionally, the baffles. Those skilled in the art will appreciate that various arrangements of the interior of the sterilisation fluid storage chamber 70 can be provided so long as sterilisation fluid is provided in fluid communication with the atomiser/vaporiser.

The sterilisation fluid storage chamber 170 may be filled with a sterilisation fluid prior to scaling of the end cap 176, or, when present, via the reloading/reloading port 180, in some embodiments the sterilisation fluid is a hypochlorous acid solution. In other embodiments alternative sterilisation fluids may be selected, for example from the following: chlorine dioxide, sodium hypochlorite, sodium hydroxide, sodium chloride, chlorine, hydrogen peroxide, photosensitisers, chloroazodin, dichlordimethylhydantoin, permanganate, alcohols (e.g. ethanol and/or isopropanol), phenols (e.g. phenol, thymol and/or chloroxyphenol), aldehydes (e.g. glutaraldehyde and/or noxythiolin) and/or acids (e.g. acetic acid, citric acid, peracetic acid and/or diperoxy dodecanoic acid) or combinations thereof.

With reference to FIG. 5 the operation of catheter sterilisation and storage device 199 is described.

The sterilisation fluid storage chamber 170 is inserted into the sterilisation unit 130. Next, a protective cap is removed from the tip 177, to allow the sterilisation fluid to exit the chamber 170, in alternative embodiments the aperture 177a may be sealed with a foil seal that is punctured by the sterilisation unit upon insertion or removed by the user. The sterilisation fluid storage chamber 170 is inserted proximal end 172 first, with the tip entering the disk aperture 146, the chamber 160 is inserted until the proximal end 172 abuts the disk 143. The sterilisation fluid storage chamber 170 is retained in the sterilisation unit, e.g. with a fixed fit. It is not always necessary to insert the sterilisation fluid storage chamber 170 prior to using the catheter steriliser, in some embodiments the sterilisation fluid storage chamber 170 may contain sufficient sterilisation fluid for multiple sterilisation cycles. In such instances the user proceeds onto attaching the catheter-receiving chamber 130 to the sterilisation unit 130 as outlined below. Where the sterilisation fluid storage chamber 170 is contains fluid for multiple sterilisation cycles the slot 135 can be used to observe the fluid in the sterilisation fluid storage chamber 170 to ensure there is sufficient.

After the catheter 101 has been used it is rinsed by the user and inserted with its distal end 104 first into the catheter-receiving chamber 110 via the distal end 113 of the catheter-receiving chamber 110, the ribs 123 guiding it in as shown in FIGS. 12a and 12b. The ribs 123 limit the extent to which the catheter 101 can be inserted into the catheter-receiving chamber 110, align it centrally within the chamber 110 and retain it in place. When fully inserted into the catheter-receiving chamber 110, the connector 107 is in contact with the ribs 123 such that the entire surface of the catheter tube 102 can be coated by the sterilisation fluid.

Next the cap 190 is placed over the open distal end 112 of the catheter-receiving chamber sealing the chamber 110. The cap is retained in place, e.g. by a fixed fit. In some embodiments corresponding engaging features may be used instead of a fixed fit, for example projections and recesses. In some embodiments an O-ring or similar seal may be provided to improve the seal between the cap 190 and catheter-receiving chamber 110. The catheter sterilisation and storage device 199 is now fully assembled.

Just prior to use of the catheter 101, the user presses the switch 154, the PCB controller 162 then directs an electric current to the piezo-electric atomiser 153 from the battery and activates the first indicator light 155a which indicates that the device is sterilising. The piezo-electric atomiser 153 oscillates at a frequency (e.g. of 142 kHz) which causes the sterilising fluid in the fibre reed 182 to be atomised/vapourised emitting a mist 180 of sterilising fluid. The mist 180 is propelled away from the atomiser 153 and is directed by the guide flange 159 towards the catheter 101. The mist 180 fills the interior of the enclosure 171 and sterilising fluid is deposited on all exposed surfaces, including the catheter tube 102. In other embodiments the sterilisation process is automatically activated when the sensor 164 detects the presence of the catheter 101.

Where present, the UV LED 163 is also activated at the same time as the piezo-electric atomiser 153.

To ensure that the catheter 101 is sufficiently sterilised, the PCB controller 162 may include a timer unit, this ensures that the sterilising fluid mist 180 is maintained for sufficient duration. During this time the first indicator light 155a is illuminated, indicating that sterilisation is occurring, once the sterilisation is complete, the first indicator light 155a is switched off and the second indicator light 155b is illuminated, indicating that sterilisation is complete, and that the user can remove the catheter 101 for use.

Once the sterilisation process is complete, the PCB controller 162 then activates the wire heater 125, this warms the catheter tube 102 to approximately body temperature thereby improving the comfort for the user. In some embodiments the warming means 125 may be activated by a separate switch or second activation of the same switch. This can ensure the catheter is warmed just prior to insertion. Next, the user can remove the catheter 101 for use by removing the cap 190 can then using the handling surfaces 108 on the funnel 105 the catheter 101 can be removed from the catheter-receiving chamber 110. By using the handling surfaces 108, the user ensures that the now sterile catheter tube 102 is not contaminated prior to use.

In a further embodiment according to the present invention an alternative arrangement of the catheter storage and sterilisation device is shown. As compared to the first embodiment, the sterilisation unit 330 is arranged alongside the longitudinal axis of the catheter-receiving chamber 310 as opposed to being arranged axially with it.

FIG. 6 shows a schematic cross-section of the catheter storage and sterilisation device 399, the catheter storage and sterilisation device comprises a sterilisation unit 330 and catheter-receiving chamber 310. As in the second embodiment, the sterilisation unit 330 and catheter-receiving chamber 310 are integrally formed.

The catheter storage and sterilisation device 399 has an overall rectangular shape, the interior of which is divided into three compartments. The first compartment is the catheter-receiving chamber 310, the catheter-receiving chamber 310 extends the full length of the device 399 and fill half the volume, it is separated from the other two chambers by a wall extending along the longitudinal length of the device 399. The electronics package 340 and sterilisation fluid storage chamber 370 fill the remaining half of the device 399 and are approximately equal in size, they are separated by a second wall normal to the longitudinal axis of the device 399. The sterilisation fluid storage chamber and catheter-receiving chamber are in fluid communication via a piezo-electric atomiser 353, which in turn is operably connected to the electronics package 340.

The catheter-receiving chamber 310 is provided with an opening at one end, proximate to the opening is a catheter holder 320, the catheter holder is formed from a plurality of ribs 323 projecting into the chamber from the interior wall. The radially inward face 324 of each rib 323 forming an engaging surface for the connector 307 of the catheter 307, the catheter 301 having the same form as that described in the previous embodiments.

The inner wall of the catheter-receiving chamber is further provided with a resistive wire heater 325, a UV LED 363 and a sensor 364. The resistive wire heater 325 is coiled around the inner wall, such that when a catheter 301 is present the heater 325 surrounds the catheter tube 302.

Arranged on the outer surface of the steriliser device 399 is a switch 354 operably connected to a PCB controller 362, which along with a battery 361, is provided within the electronics package 340.

There is also provided a cap 390 shaped to seal the opening in the catheter-receiving chamber 310.

In use, after the catheter 301 has been used it is rinsed by the user and inserted into the catheter-receiving chamber 310 distal end 304 first, such that the catheter holder 320 engages with the connector 307 on the catheter 301, this allows the entire surface of the catheter tube 302 to be coated in sterilisation fluid.

Next the cap 390 is placed into the opening to seal the catheter-receiving chamber 310, it is held in place by a fixed fit. In some embodiments corresponding engaging elements on the cap and catheter-receiving chamber may be used to secure the two components. In some embodiments a seal, such as an O-ring may be provided to improve the seal between the two components. The catheter sterilisation and storage device 399 is now fully assembled.

Just prior to use of the catheter 301, the user presses the switch 354, the PCB controller 362 then directs an electric current to the piezo-electric atomiser 353 from the battery 361 and activates the first indicator light which indicates that the device is sterilising. The piezo-electric atomiser 353 oscillates at a frequency (e.g. of 142 kHz) which causes the sterilising fluid in the sterilisation fluid storage chamber 370 to be atomised, emitting a mist of sterilising fluid. The mist is propelled away from the atomiser 353 and is directed towards the catheter 301. The mist fills the interior of the catheter-receiving chamber 310 and sterilising fluid is deposited on all exposed surfaces, including the catheter tube 302. In other embodiments the sterilisation process is automatically activated when the sensor 364 detects the presence of the catheter 301.

Where present, the UV LED 363 is also activated at the same time as the piezo-electric atomiser 353.

To ensure that the catheter 301 is sufficiently sterilised the PCB controller 362 includes a timer unit, this ensures that the sterilising fluid mist 380 is maintained for sufficient duration. During this time the first indicator light is illuminated, indicating that sterilisation is occurring, once the sterilisation is complete, the first indicator light is switched off and the second indicator light is illuminated, indicating that sterilisation is complete,

Once the sterilisation process is complete, the PCB controller 362 then activates the wire heater 325, this warms the catheter tube 302 to approximately body temperature thereby improving the comfort for the user. Next, the user can remove the catheter 301 for use by removing the cap 390 can then using the handling surfaces 308 on the funnel 305 the catheter 301 can be removed from the catheter-receiving chamber 310. By using the handling surfaces 308, the user ensures that the now sterile catheter tube 302 is not contaminated prior to use.

In a further embodiment according to the present invention an alternative arrangement of the catheter storage and sterilisation device 499 is shown. As compared to the previous embodiments, the device comprises a plurality of catheter-receiving chambers 410.

With reference to FIGS. 7 and 8, the catheter storage and sterilisation device 499 of this embodiment has a cylindrical outer body 430 with a rounded edge 432 to the base 433. The upper edge 434 is beveled and the upper face 435 contains three apertures 436 for multiple (in this case three) respective catheter receiving chambers 410 a-c. On the edge of each aperture, a portion 437 is recessed to improve the ease at which the user can remove a catheter 401 from each aperture.

The device 499 is also provided with a cap 490 and in this embodiment the edge of the cap is shaped to correspond to the beveled edge of the upper edge of the device body.

The device (in this case on the curved face of the body) is provided with a switch 454 operably connected to the electronics package 440, as described in greater detail below.

FIG. 8 shows a cross-section of the device along the line A-A of FIG. 7. Each catheter-receiving chamber 410 is similar to that of the second embodiment. Each chamber 410 is cylindrical with a closed bottom, as noted above, the top end is open, so as to allow a catheter 401 to be inserted.

One catheter-receiving chamber 410 will be discussed in detail, but it will be appreciated that each chamber 410a,b,c is identical.

Proximate to the opening 436, on the interior surface 422 of the catheter-receiving chamber 410 are provided three ribs 423 arranged equidistantly around the circumference, the ribs 423 extend parallel to the rotational axis of the catheter holder and project radially inwards. The radially inward facing edges of the ribs 423 define engaging surfaces 424 which engage with the catheter 401, as such that a circle defined by the engaging surfaces is approximately equal to the outer diameter of the connector 407 (described below). These ribs form a catheter holder 420.

Further arranged on the interior surface 422 is a resistive wire heater 425, the wire heater 425 is coiled around the interior surface between the catheter holder 420 and the piezo-electric atomiser 453, arranged at the end opposite the opening 436. In some embodiments all of the catheter-receiving chamber may be fluidly connected to a single piezo-electric atomiser. Similarly, a single heater could heat all the chambers.

Also arranged proximate to the opening is a sensor 464 configured to detect the presence of the catheter 401.

Once again, this embodiment of the invention includes the optional UV LED 463, which is arranged proximate to the piezo-electric atomiser 453.

On the opposing side of the piezo-electric atomiser 453 to the catheter-receiving chamber 410 is a sterilisation fluid storage chamber 470, as described in the previous embodiments, the sterilisation fluid storage chamber 470 is provided with a port to allow the chamber to be filled/refilled with sterilisation fluid. In some embodiments a single sterilisation fluid storage chamber is provided to supply all of the catheter-receiving chambers. In other embodiments each catheter-receiving chamber is provided with its own sterilisation fluid storage chamber.

Arranged below the sterilisation fluid storage chamber 470 is the electronics package which comprises a PCB controller. In some embodiments the electronics package further comprises a battery which is rechargeable via a USB cable. In other embodiments the device 499 is mains powered.

In use, after the catheter 401 has been used it is rinsed by the user and inserted into one of the catheter-receiving chambers 310a,b,c distal end first, such that the catheter holder 420 engages with the connector 407 on the catheter 401, this allows the entire surface of the catheter tube 402 to be coated in sterilisation fluid.

Next the cap 490 is placed over the upper face 435 to seal the catheter-receiving chambers 410, it is held in place by a fixed fit. In some embodiments corresponding engaging elements on the cap and outer body 430 may be used to secure the two components. In some embodiments a seal, such as an O-ring may be provided to improve the seal between the two components.

Just prior to use of the catheter 401, the user presses the switch 454, the PCB controller then directs an electric current to the piezo-electric atomiser 453 from the battery or mains supply and activates a first indicator light which indicates that the device is sterilising. The piezo-electric atomiser 453 oscillates at a frequency of 142 kHz, this causes the sterilising fluid in the sterilisation fluid storage chamber 470 to be atomised emitting a mist of sterilising fluid. The mist is propelled away from the atomiser 453 and is directed towards the catheter 401. The mist fills the interior of the catheter-receiving chamber 410 and sterilising fluid is deposited on all exposed surfaces, including the catheter tube 402. In other embodiments the sterilisation process is automatically activated when the sensor 464 detects the presence of the catheter 401.

Where present, the UV LED 463 is also activated at the same time as the piezo-electric atomiser 453.

To ensure that the catheter 401 is sufficiently sterilised the PCB controller includes a timer unit, this ensures that the sterilising fluid mist is maintained for sufficient duration. During this time the first indicator light is illuminated, indicating that sterilisation is occurring, once the sterilisation is complete, the first indicator light is switched off and the second indicator light is illuminated, indicating that sterilisation is complete.

Once the sterilisation process is complete, the PCB controller then activates the wire heater 425, this warms the catheter tube 402 to approximately body temperature thereby improving the comfort for the user.

Next, the user can remove the catheter 401 for use by removing the cap 490 and then using the handling surfaces 408 on the funnel 405, the catheter 401 can be removed from the catheter-receiving chamber 410. By using the handling surfaces 408, the user ensures that the now sterile catheter tube 402 is not contaminated prior to use.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Except in Examples, or where otherwise explicitly indicated, all numerical quantities in this description specifying amounts of materials, device dimension, and the like, are to be understood as modified by the word “about.”

Unless otherwise indicated, each chemical or composition referred to herein should be interpreted as being a commercial grade material which may contain the isomers, by-products, derivatives, and other such materials which are normally understood to be present in the commercial grade.

The one or more embodiments are described above by way of example only. Many variations are possible without departing from the scope of protection afforded by the appended claims.

1. A catheter storage device comprising a catheter-receiving chamber for receiving a catheter and a catheter warming means, the catheter warming means configured to warm the catheter.

2. A catheter storage device according to claim 1 wherein the catheter-receiving chamber is elongate.

3. A catheter storage device according to claim 1 or 2 wherein the catheter-receiving chamber is tubular.

4. A catheter storage device according to any preceding claim wherein the catheter warming means is a heater.

5. A catheter storage device according to claim 4 wherein the heater is a resistive wire.

6. A catheter storage device according to claim 5 wherein the resistive wire is wound helically around the catheter-receiving chamber.

7. A catheter storage device according to claim 5 or 6 wherein the resistive wire is arranged on the interior wall of the catheter-receiving chamber.

8. A catheter storage device according to any preceding claim further comprising a switch configured to activate the heater.

9. A catheter storage device according to any preceding claim further comprising a sterilisation device.

10. A catheter storage device according to claim 9 wherein the device is configured to sterilise the catheter prior to warming the catheter.

11. A catheter storage and sterilisation device according to any preceding claim further comprising a urinary catheter.

12. A catheter storage and sterilisation device according to claim 11 wherein the catheter is an intermittent catheter.

13. A catheter storage and sterilisation device according to claim 11 or 12 when dependent upon claim 9 wherein the sterilisation device is arranged proximate to an insertion end of the catheter.

14. A catheter storage and sterilisation device according to any preceding claim further comprising an external housing, the external housing having an elongate shape.

15. A catheter storage and sterilisation device according to any preceding claim wherein an exterior wall of the catheter-receiving chamber comprises an exterior wall of an external housing of the device.

16. A catheter storage and sterilisation device according to any preceding claim further comprising a sterilisation unit comprising one or more of the following: the sterilisation fluid storage means, a sterilisation device, a battery, a PCB controller, one or more indicators.

17. A catheter storage and sterilisation device according to claim 16 wherein an external wall of the sterilisation unit comprises an external wall of an external housing of the device.

18. A catheter storage device according to any preceding claim wherein the catheter storage device comprises a plurality of catheter-receiving chambers.

19. A method of warming a catheter; the method comprising; inserting a catheter into a catheter storage device comprising a catheter warming means, activating the catheter warming means, and warming the catheter.

20. A method of warming a catheter according to claim 19 wherein the catheter storage device is a catheter storage device according to any of claims 1 to 18.

21. A method of warming a catheter according to claim 19 or 20 wherein the catheter storage device further comprises a sterilisation device; and the method further comprises the step of sterilising the catheter.

22. A method of warming a catheter according to claim 21 wherein the step of sterilising the catheter is performed prior to the step of warming the catheter.
